(19) United States
(12) Patent Application Publication
(10) Pub. No.: US 2012/0122966 A1
(43) Pub. Date: May 17, 2012

(54) USE OF ORGANIC DYES AS ANALGESIC AGENTS

(76) Inventor: Mikhail Vladimirovich Kutushov, Moscow (RU)

(21) Appl. No.: 13/138,878

(22) PCT Filed: Apr. 14, 2009
(86) PCT No.: PCT/RU2009/000178

§ 371 Date: Jan. 4, 2012

(51) Int. Cl. A61K 31/136; A61K 31/352; A61P 25/04; A61K 31/196

(52) U.S. Cl. 514/454; 514/641; 514/574

(57) ABSTRACT

An organic dye from the group of crystal violet, gentian violet, malachite green oxalate and rhodamine 6G used as an analgesic agent in the form of a solution with the agent dosage ranging from 0.000001 mg to 10 g per intake. It is preferable to take the agent at least two times a day. In particular cases of use the agent is administered by mouth in the form of an aqueous solution before or after meals, or it is administered per rectum in the form of an aqueous solution, or a 1% solution of the agent is administered intravenously, or the agent is used in the form of a 1-5% ointment, or the agent is administered per rectum in the form of suppositories, and when arresting cancer pain the agent is injected into the tumor (under the tumor) or into a metastasis. Herein, one uses as analgesic agents widely available and inexpensive substances that are non-toxic, have no allergic effects and cause no addiction (dependence) when used in pharmaceutically acceptable doses; the treatment time is reduced, treatment efficiency is enhanced, and pain relief is provided while preserving patients' functional activity, using the minimum arsenal of analgesic agents with different effects, and completely eliminating the need for opiates.

### BACKGROUND OF THE INVENTION

1. Field of the Invention

This invention relates to medicine, and specifically to analgesic medicinal preparations for treatment of acute, chronic and nociceptive pain.

2. Discussion of Related Art

Pain is the most frequent reason patients seek medical care. Pain syndrome is one main reason of lower quality of patient's life in most illnesses, and it can dominate in his/her clinical picture, it can be the main, and sometimes the only symptom of disease. The ability to feel pain is the most important defense mechanism which ensures living organisms' survival, learning and adaptation to changing environmental conditions.

Burning and darting pain is called neuropathic pain.

Pain related to activation of pain receptors (nociceptors) after tissue damage and commensurate with the degree of the tissue damage and the duration of the effect of damaging factors is called nociceptive pain. Afferent nociceptive peripheral nerves have small-diameter primary fibers which have receptors in various organs and tissues. Substances (algogenes) synthesized or freed as a result of pathologic damage of tissues stimulate the activity of nociceptive nerve endings. Pain stimulations are perceived by free nerve endings (nociceptors). The sensation of pain is associated with the appearance of electric activity, depolarization of sensitive nerve endings.

Analgesic agents, or analgesics (from the Greek algos—pain and an—without) are medicinal agents with the specific capacity to relieve or eliminate the feeling of pain. Analgesic (pain relieving) action can be provided not just by strictly analgesics, but also by other substances that pertain to various pharmacologic groups.

Known is novocain, an anesthetic agent widely used in surgery (See Shchelkunov, V. S., “ [symbol] [symbol] [symbol] ” [Peridural Anesthesia]. Leningrad, “Meditsina” Publishing House, 1976, p. 3-37).

The agent's disadvantages are incomplete and insufficiently fast pain relief, and also the fact that the body gradually becomes addictive to it.

The CNS is most strongly suppressed by a group of general anesthetic preparations (narcotic drugs). Next are soporifics. The action of this group is not as strong as that of general anesthetics. Then, in the descending order of strength, are alcohol, anticonvulsants and antiparkinsonian preparations.

Known is a prolonged-action anesthetic preparation that has as the active principle a β-form of tetracaine—methylcellulose which insures prolonged action of anesthetics, and a pharmaceutical solvent used for preparation of eye drops (See Patent Reference RU No. 2038076).

One disadvantage of the prolonged-action anesthetic composition is its narrow scope of use, as a local anesthetic for ophthalmology.

Also known is a prolonged-action anesthetic composition that uses trimecaine as the active principle, and collagen in acetic solution as the prolonged-action agent (See Patent Reference SU No. 880426).

However, this composition has not been widely used in medicine due to its limited scope of use (dentistry).

There is also a group of preparations that have suppressing effect on the psycho-emotional sphere—central-acting psychotropic agents. Antipsychotic neuroleptics are the strongest group; tranquilizers are the second, less potent group; and general sedatives are the third group. There is a type of general anesthesia—neuroleptanalgesia. For this type of analgesia one uses mixtures of neuroleptics and analgesics. This is a condition of anesthesia with preserved counsciousness. Currently, neuroleptoanalgesics are widely used. This is a mixture of neuroleptics and analgesics. Droperidol can be used as a neuroleptic, and phentamine (several hundred times stronger than morphine) can be used as an analgesic. This mixture is called talomonal. One can use aminasine instead of droperidol, and promedol instead of phentamine; promedol's effect will potentiate a tranquilizer (seduxen) or clonidine. Even analgin can be used instead of promedol. Currently, non-narcotic analgesics such as amidopyrine, phenacetin, analgin, etc. are used. However, they do not have prolonged pain relieving effect, and they also can have side effects. In experiments on animals, the possibility of carcenogenic effect of amidopyrine (especially with prolonged use) was discovered, as well as its damaging effect on the hematopoietic system. Phenacetin can also have nephrotoxic effect. Because of this, these preparations have limited use, and a number of finished medicinal agents that contain these preparations (amidopyrine solutions and granules; pyraminal, diaphein and analphen tablets, amidopyrine+phenacetin tablets, etc.) were deleted from the nomenclature of medicinal agents. At the same time paracetamol has been used more widely. Most often one uses analgesics from the narcotic series—opiates, phentamine, morphine, and promedol. In case of severe pain in cancer patients in severe stages with multiple metastases, narcotic analgesics are used as a rule. When introduced into a body, morphine, an exogenous analgesic (as well as other opiates and opioids whose structure is close to it), interacts with the same binding areas (receptors) whose function is to bind endogenous analgesic compounds—enkephalins and endorphins (See “ [symbol] [symbol] [symbol] ” [Handbook of Drugs], http://pharm.stirol.net).

Morphine, one of the strongest narcotic preparations, is used as the prototype. Morphine belongs to the group of morphinane alkaloids and to the group of isoquinoline alkaloids. Its chemical formula is C₁₇H₁₉N0₃. The morphine molecule has 5 asymmetric carbon atoms. Therefore, morphine has a lot of isomers, including α-, β- and γ-isomers. Physical properties of the isomers are somewhat different, especially their optical rotation indices. Colorless prismatic crystals with bitter taste. Morphine is water-soluble but alcohol-insoluble. Color reactions. Morphine reacts with concentrated nitric acid (blood-red, passing into orange-yellow). Red-yellow stain with Erdman's reagent (concentrated sulfuric acid with concentrated nitric acid). Violet stain with Fred's reagent (concentrated sulfuric acid with molybdic acid). Violet stain with Mandelin's reagent (concentrated sulfuric acid with vanadic acid). Violet stain with Markey's reagent (concentrated sulfuric acid and formaldehyde). Pharmacologically, morphine is a complete agonist of μ-opioid receptors and it acts on both of their subtypes. Morphine's agonistic effect on opiate receptors is accompanied by lower level of consciousness, the sense of warmth, drowsiness and euphoria (when the preparation is administered for the first time, some people develop dysphoria). Acute toxicity of morphine LD50=170 mg/kg. (Wikipedia, Internet).

Brown University's researchers discovered a protein that is affected by morphine causing narcotic addiction. It turned out that this protein is guanylate cyclase enzyme. Studies on mice demonstrated that morphine introduced into an animal's body damages precisely the protein, and the effect lasts for 24 hours after administering a single dose of morphine. Guanylate cyclase is contained in synapses—junctions of two neurons, an inhibitory and a dopamine neuron in this case. Dopamine neurons release dopamine—a pleasure hormone which in a healthy body is released in response to natural positive stimuli, for instance, following a meal, after sex, etc. Normally, an inhibitory neuron stops dopamine release. Morphine blocks the signal from the inhibitory neuron; as a result, the dopamine neuron continues “discharging” new portions of dopamine. Thus, a narcotic disables the brain's natural “brakes”. Morphine damages the brain's natural “brakes”.

(See Internet. Article “ [symbol] [symbol] [symbol] [symbol] ‘ [symbol] ’ [symbol] [symbol] ” [Morphine Damages Brain's Natural “Brakes”] http://www.polit.ru).

Morphine must be used under strict medical supervision. Morphine's characteristic feature is its inhibiting effect on the central nervous system and, as a consequence, the development of drug addiction. In the course of treatment using the preparation, rhythm disturbance, heart failure and lower arterial pressure may develop. In case of severe heart rhythm disturbances morphine treatment must be stopped immediately. The use of morphine is contraindicated in cases of severe CNS and rhythm disturbances.

It is believed that narcotic analgesics and opiates are the main therapeutic agent for severe pain syndromes.

Besides, in addition to them it is often necessary to use medicinal preparations from other pharmacologic groups that can potentiate the pain relieving effect of narcotic analgesics.

One of the closest analogues are preparations based on opiate derivatives, morphine derivatives—heroin. Heroin—diacetylmorphine, a 3,6-diacetyl derivative of morphine, or diamorphine (diamorphine, according to BAN). A semi-synthetic opioid narcotic. Chemical formula C₂₁H₂₃N0₅. Pharmacological action—analgesic (narcotic). Has the properties of an opiate receptor agonist-antagonist. Stimulates μ-receptors and blocks κ-receptors. Inhibits the CNS, and has analgesic, hypnotic and cough-depressing effect.

Currently, researchers (physicians and pharmacologists) are considering the prospect of replacing morphine with heroin.

The preparation has pronounced analgesic activity for relieving pain in many pain syndromes. However, heroin is not effective in cases of excruciating pain caused by bone metastases. The preparation has a fairly toxic effect when administered in large doses, and it contributes to addiction, which also limits its use in large doses. Depression of consciousness, from drowsiness and sopor to coma, is possible. The specific symptom is a sharp miosis (pinhead-size pupils) and decreased or absent reaction to light. As the condition gets more severe and brain hypoxia increases, the pupils are dilated; reaction to light is decreased or absent. The most dangerous for the victim is respiratory distress. It is manifested in the form of lower respiratory rate. In case of pronounced intoxication the respiratory rate is 4-6 per minute. Convulsive, jerky and noisy breathing, inhibited hemodynamics, lower arterial pressure, slow thread pulse, increased clinical manifestations of left ventricular failure. The most dangerous for the victim are non-coronary pulmonary edema, increasing heart failure, and adult respiratory distress syndrome. The most frequent cause of death is increasing lung edema as the result of heart failure (See “ [symbol] [symbol] [symbol] [symbol] [symbol] [symbol] [Urgent States of Opiate Addiction], “ [symbol] [symbol] ” [“Practicing Physician” magazine], No. 5, 2006).

Known are analgesics in the form of a synthetic analgesic—moradol, and general anesthetics, wherein during surgery clonidine and contrycal are added, and during the post-surgery period water-soluble acetylsalicylic acid salt is administered in addition to the above preparations (See Patent Reference RU 2016586).

Some disadvantages of this solution are the difficulty of use and toxic effect of the preparations, high cost of the course of treatment, and the possibility of allergic reactions and gastrointestinal tract disorders.

Known is the use of organic dyes in microbiology and histology as a means for postmortem diagnostics. To do this, tissues and cells are stained using organic dyes. In the process, tinctorial properties of microbes, cells, organelles and tissues are changed. Tinctorial properties are the capacity to assimilate dyes and get the characteristic stain. The most important for identification is the use of complex (differentiating) methods, and first of all the Gram's method which makes it possible to differentiate gram-positive and gram-negative bacteria. When staining using this method, gram-positive bacteria are stained indigo blue, and gram-negative bacteria are stained burgundy red, which reflects the differences in the structure of cell walls of the two groups of bacteria (See “ [symbol] [symbol] [symbol] [symbol] [symbol] [symbol] [symbol] [symbol] [symbol] ” (KMAX) [Clinical Microbiology and Antimicrobial Chemotherapy (CMAC)], 2000, Vol. 2, No. 2—“ [symbol] [symbol] [symbol] ” [Diseases and Activastors]—I. V. Smirnov—“ [symbol] [symbol] [symbol] [symbol] [symbol] [symbol] ” [Activators of Bacterial Infections in Humans]). Currently, the method for differential diagnostics of stomach tumors using methylene blue is used. In the early 20^{th} century, the method for diagnostics of amyloidoses using intravenous injection of colloidal dyes (Congo red) was used.

### DETAILED DESCRIPTION OF INVENTION

One objective of this invention is to provide a medicinal agent that has a pronounced analgesic effect in various long-term chronic pain syndromes, also including oncologic diseases, as well as an analgesic agent for general anesthesia that does not have side effects.

The technical result that provides the solution of the objective is the use as an analgesic agent of widely available and inexpensive substances that are non-toxic, have no allergic effects and cause no addiction (dependence) when used in pharmaceutically acceptable doses; reduced treatment time; enhanced treatment efficiency, including providing pain relief while preserving patients' functional activity; the use of the minimum arsenal of analgesic agents with various action targets; and complete elimination of the need for opiates.

This invention is used as an analgesic agent of an organic dye from the group of crystal violet, gentian violet, malachite green oxalate and rhodamine 6G in the form of a solution with the dosage ranging from 0.000001 mg to 10 g per intake.

It is preferable to take the analgesic at least two times a day. In particular cases of use the agent is administered by mouth in the form of an aqueous solution before or after meals, or per rectum in the form of an aqueous solution, or a 1% solution of the agent is administered intravenously, or the agent is used as a 1%-5% ointment, or the agent is administered per rectum in the form of suppositories, and when arresting cancer pain the agent is injected into the tumor (under the tumor) or into a metastasis.

As stated in the application, among such analgesic agents there are some organic dyes belonging to substances that can change optical and electromagnetic polarization (linear, circular, etc.) in live tissue and serve as a sort of coherent radiation generators and “lenses”, like a laser. According to M. V. Kutushov's theory, the main routes of information transmission are protein structures operating based on the fiber optics principle. Protein strings have direct relation to mechanisms responsible for pain (See book “Pak — [symbol] [symbol] [symbol] [symbol] ” [Cancer: an Instrument for Learning the Phenomenon of Life], “Dzhangar” Publishing House, Elista, 2001, p. 39, and book “ [symbol] ” [Cancer is Curable], M. V. Kutushov, Sekachev Publisher, Moscow, 2005, p. 141). The dyes can serve as “gates” in the pain pulse path, or they can form crystalline hydrates that interrupt pain impulses in pain conducting paths.

According to M. V. Kutushov's theory, because when the molecular dissymmetrizing mechanism breaks down and when malignant cells anisotropy is lost and polarization in them is disturbed, organic dyes change tinctorial properties of malignant cells while alive, and restore the dissymmetry, anisotropy and polarization; in doing so, in therapeutic doses they have practically no effect on healthy cells but provide an opiate-like effect. The opiate-like effect can be due to concurrent light absorption by receptors and dyes. The proposed dyes can act as the agonist of μ-opioid receptors, staining conducting beige structures in pathologically changed tissues, which results in long-term analgesic effect.

The analgesic effect is due to the fact that organic dyes give back to proteins that are part of the conducting system (pain, tactile sensation, etc.) the capacity to coherently absorb and luminesce in the normal-narrow-waveband. Among such substances are organic dyes that have a particular spatial structure. The absence of side effects in the dyes is due to the fact that normal protein structures and cells, having anisotropy, do not react to the presence of organic dyes. The spatial inversion structure of cell structures contributes to the change of tinctorial properties. In case of degeneration of cell structures and protoplasm, it is in them that spatial disturbances of protein structures occur first of all. Depending on their structure, organic dyes have the capacity to absorb and luminesce in a certain waveband and to oscillate. Therefore, by changing tinctorial properties of pathologically changed structures (including also the structures responsible for pain) the dyes normalize metabolism, return the structures to their normal state, and relieve pain.

Organic dyes to some extent have the capacity to absorb and generate radiation. Generation spectrums of most dyes have a series of sharp equidistant lines the distance between which depends on the properties of the resonator. The resonator has a number of reflecting surfaces. If a cell itself and its structures are allowed as resonators, then the resonating properties will be changing depending on the norm and pathology. In particular, in case of cancer cell and tissue structures, tissue anisotropy and cytoplasm dissymmetry are most grossly disturbed; as a result, isotropy prevails in them. It is known from physics that when a material loses its anisotropy, its thermal, mechanical, electromagnetic, optical, etc. properties change. This law is also in effect in living matter which in essence comprises aperiodic liquid crystals. It is these very disturbances that occur in malignization. Administration of acceptable dyes facilitates restoration of anisotropy and as a result the involution of malignant cells. In addition, restoration of anisotropy in cancer homeostasis and metastases changes the medium pH in the cancer focus, thus facilitating the disappearance of the so-called “alkaline pains” typical in terminal cancer patients.

The proposed dyes have high absorptivity in the visible spectrum. Therefore, the dyes interacting with the changed structures give back to the structures their original narrow absorption spectrums or, to put it differently, the so-called equidistant properties. Thus, one can conclude that they, by themselves or by joining with cell structures and forming clusters with water clathrates (crystalline hydrates), can damp the wavelength that cancer structures “use” for their vital activity and thus cause their death or involution.

Most organic dyes can luminesce, which is a therapeutic factor. Symmetric and asymmetric dyes are therapeutic agents in the same extent for treatment of various diseases, including as analgesic preparations. In pathologically changed tissues and cells where isotropy prevails, organic dyes can serve as generators of coherent radiation. Organic dyes can also generate photons in a narrow waveband. The most important property of dyes is an extremely high cross-section of the limit amplification of luminescence—five orders of magnitude higher than the ruby R-line (See “ [symbol] [symbol] [symbol] [symbol] ” magazine, vol. 95, No. 1, 1968, May, pp. 45-50. B. I. Stepanov, A. N. Rubinov, “ [symbol] [symbol] [symbol] [symbol] [symbol] [symbol] [symbol] [symbol] [symbol] [symbol] ” [Optical Quantum Generators Based on Organic Dye Solutions]). Based on universal optical properties and the property to stain damaged and healthy tissues and cells differently, the proposed organic dyes were used for treatment of malignant tumors, somatic mental diseases and for pain relief.

To amplify the therapeutic effect, hydrogen (H) atoms in the dyes are replaced in the cyclical section with radicals (CH3 or C2H5), which changes considerably the dyes capacity and the spectral position of lines (Stepanov, B. I., Rubinov, A. N., “ [symbol] [symbol] [symbol] [symbol] [symbol] [symbol] [symbol] [symbol] [symbol] ”, “ [symbol] [symbol] [symbol] [symbol] ”, Haye, T. 95, [symbol] 1 1968. [symbol] [Optical Quantum Generators Based on Organic Dye Solutions, “ [symbol] [symbol] HayK” magazine, vol. 95, No. 1, 1968, May]). Currently, organic dyes are used in lasers, forensic medicine, for biologic research, in microscopy for intravital staining of cell nuclei, for staining of erythrocytes and ascarid eggs, and also as a medicinal agent when treating aquarium fish from infectious diseases, etc.

The following is a list and formulas of organic dyes used for treatment of somatic mental diseases and for analgesic preparations.

1. Gentian violet and crystal violet.

2. Malachite green oxalate.

3. Rhodamine 6G.

Studies have demonstrated that medicinal agents in the form of the listed substances have an analgesic effect. In doing so, they excite μ-receptors and block κ-receptors. The body's antinociceptive system (which suppresses pain propagation and appreciation) gets activated. It can block algesic impulsing at all levels of the nociceptive system—from receptors to its central structures.

They also restore disturbed homeostasis in cases of somatic diseases and mental disorders. This was confirmed in laboratory instrument examinations by performing patients' blood tests, instrumental study methods (CT, NMRI, X-ray studies, etc.), and measuring tumor dimensions before and after taking appropriate organic dye-based preparations. They can be administered by mouth, per rectum or intravenously if they are dissolved, strained, filtered, recrystallized and sterilized.

Organic dye-based medicinal preparations can be taken by mouth in the form of powder or tablets with or after meals, per rectum in the form of suppositories, externally in the form of suspensions and ointments; in sterile form they can be administered intravenously and intramuscularly, locally into the tumor or into an inflammatory degenerative pathological focus, and also intracavitary. And their use in a wide dosage range (from 0.000001 mg to 10 g) does not cause allergic reactions or have other side effects, and their efficiency (in treating appropriate diseases) is higher than for instance the efficiency of the preparation selected as the prototype.

Results of their use for treatment are corroborated by examples 1-10.

### EXAMPLE 1

Male patient V., 54 years old. Diagnosis: poorly differentiated pulmonary adenocarcinoma. MTS [expansion not given; possibly metastases] to the brain and skeleton bones. Cachexia. Pain syndrome. Relatives state that over the last two months the patient has been receiving 5.0 ml of heroin intravenously three times a day. Otherwise, he notes resumption of pronounced excruciating pain. In CT, several tumor-like masses in the mediastinum and left lung, average size 3.9×3.6×3.2 cm. Grave condition, dyspnea, cyanosis, adynamia, and sensations of pain, getting stronger when coughing. Treatment was prescribed that included the use of medicinal preparations with antineoplastic action—an organic dye, a 1% gentian violet solution. The first course of treatment using the preparation—15 drops of a 1% solution in a glass of water three times a day by mouth, additionally a microclyster—10 drops of a 1% solution in 30 ml of water at lunch, and q.h.s. a 1% suppository with the preparation. For a week, essentially no change of patient's condition, however the patient was noting that after the sessions there was for several hours a substantial improvement of the pain syndrome. On the third week of treatment with the preparation the patient's condition improved. Three months after the start of treatment with the preparation, no MTS in the brain were observed in CT. The lung tumor size dropped to 2.1×2.2×2.2 cm. After six months of treatment the tumor size dropped to 0.3×0.4×1.0 cm. No brain metastases are observed, dyspnea abated, the pain syndrome is practically absent. Blood tests against the background of treatment have normalized.

### EXAMPLE 2

Male patient T., 29 years old.

Three years ago, the left kidney was removed, and preventive chemotherapy was administered for renal cell carcinoma. In a CT examination, a 2.5×2.4×2.3 cm tumor-like mass in the right kidney projection, three round tumor-like masses from 2.6 to 2.3 cm in diameter in the left lung, and numerous bone metastases. Grave condition, dyspnea, cyanosis, adynamia. Complaints of excruciating bone pain. Takes 3 ml of morphine 3-4 times a day. Treatment prescribed, including the use of an antitumor medicinal preparation. The preparation, crystal violet, was administered by mouth as a potion—16 drops of a 1% solution three times a day, and as microclysters. After 7 days of treatment, dyspnea abated, and the pain syndrome disappeared. Blood tests against the background of treatment have normalized over 3 weeks. Then, the course was repeated using crystal violet preparation which was administered into rectum in the form of 1% suppositories and by inhaling, for 12 months with half-month long breaks. During the therapy the right kidney tumor and lung metastases disappeared. No pain. Weight restored.

### EXAMPLE 3

Male patient L., 54 years old. Diagnosis: bladder cancer. Metastases in pelvic bones. Three years ago bladderectomy was performed for cancer. Complaints of pain, bloody urine, frequent urination and pelvic bone pains. In CT, a 2×2×2 cm tumor-like mass in the projection of the mouth of the right ureter and a 1.5×1.1×0.9 cm in the projection of the mouth of the left ureter. In the wing of the left illium two tumor-like masses with irregular contours, 1.5 to 2.5 cm in diameter. Medium severity condition, skin integument pallor, blood in urine. According to the patient and his relatives, he is taking 5.0 ml of morphine three times a day. Began treatment with the preparation—20 drops of a 1% solution of malachite green oxalate in half a glass of water by mouth and in the form of microclysters. On the sixth day, pain in the area of metastases got duller, and on the tenth day it disappeared completely. The patient declined taking narcotic analgesics. The course had lasted for 4 months. Over the course of treatment the tumor size decreased to 0.2×0.2×0.2 cm. Pelvic bone metastases decreased by 1.5 cm. Biphosphonates were prescribed for replacement therapy.

On the fourth week, blood tests against the background of treatment have normalized. A complete course of treatment with the preparation had been conducted for 6 months. During the therapy the bladder tumor and pelvic metastases disappeared. The pain did not return. Weight restored.

### EXAMPLE 4

Male patient D., 44 years old. Diagnosis: metastasizing melanoma of the right forearm. Metastases to the liver and right eye. Three years ago melanoma resection was performed. A year and a half later gripes appeared in the right eye. Eye enucleation for metastasis was performed. In the examination of the internal organs, a 4×5×4 cm metastasis in the liver was detected. At the same time, relapse in the surgery area appeared. Complaints of severe pain in the liver and pain in the relapse area. Objectively: medium severity condition, skin integument pallor, a purple tumor-like mass on the right forearm with a 4×5×5 cm pronounced edema. According to the patient, he is taking 3.0 ml of morphine four times a day. Began treatment with the preparation according to the following regimen: 5 ml of a 0.000001% sterile solution of malachite green oxalate was injected under the tumor base. Later, a 2% ointment with the same preparation was applied to the skin surface. Two times a day, 500 mg of malachite green oxalate diluted in 500 ml of saline was administered intravenously. Also, 5 g of malachite green oxalate was diluted in water and administered by mouth, as well as in the form of microclysters. On the sixth day, pain in the area of metastases became duller, and on the tenth day it disappeared completely. The patient declined taking narcotic analgesics. The course had continued for 3 months. During the course of treatment the tumor in the forearm area decreased to 0.2×0.2×0.2 cm. The liver metastasis decreased by 1.5 cm. On the fifth day after the start of treatment the patient declined taking narcotics. Blood tests against the background of treatment had normalized on the 4^{th} week. A complete course of treatment with the preparation had been conducted for 6 months. During the course of the therapy the bladder tumor and pelvic' metastases disappeared. The pain did not return. Weight restored.

### EXAMPLE 5

Male patient Z., 62 years old.

In June 2002 the patient felt (repeatedly) abdominal pain after meals and a slight weight loss. In magnetic resonance tomography of the abdominal cavity, a tumor in the pancreas area was detected. No filling defect in the stomach lumen. A non-homogeneous gastric contents signal. The pancreas size 2.2-2.1-1.6 cm, with a slightly wavy fuzzy contour and a non-uniform diffuse structure. A slightly dilated (0.4 cm) pancreatic duct. In the head area, a 5.2×36 cm tuberous non-uniform mass with fuzzy contours is detected.

The spleen is not enlarged, its structure is uniform, with a distinct even contour. Regional lymph nodes are not enlarged.

Diagnosis: an MR picture of a three-dimensional mass in the pancreatic head area. Chronic pancreatitis.

Neck lymph node biopsy was performed.

Diagnosis: Susp. C-g of the pancreas, Mtz to neck lymph nodes.

Histological study: A lymph node with Mtz of a poorly differentiated malignant tumor, most probably of epithelial nature, in the state of subtotal necrosis.

To make the diagnosis more precise, the second study was conducted.

Study result: the lymph node tissue is practically completely necrotized; there is only a narrow tissue strip under the capsule. There is a group of large cells filling up the marginal sinus. Cell nuclei are rounded, with pedicular chromatin and 2-3 basophilic nucleoluses. Extensive and amphiphilic cytoplasm. In individual vacuoles there is positively stained mucus. Numerous mitosis figures are encountered. Histological study results: tumor cells pronouncedly express papuntokeritin (MNF 116), EMA, SDKh2, and weekly express CK7. Negative expression of mesothelin CK20, TTF-1, CK 5/6, and PSA.

Conclusion: metastasis of poorly differentiated adenogenic cancer. The most probable cause of metastasizing is pancreas.

P.S. Antipherative activity of tumor cells is around 70%.

After studies had been conducted it was found that the patient has pancreas head blastoma with metastases in neck lymph nodes on the left.

Started chemotherapy. After three weeks the condition began rapidly deteriorating, accompanied by extreme weight loss—from 95 kg to 72 kg. Pain became considerably more severe. Stomach pain occurs before having a meal, i.e. after having a meal the pain is lower. At the same time stool was lightly yellow—very light color. The patient had been taking an analgesic—ketanol—infrequently, but lately has been taking it two times a day. Then he switched to taking morphine, 2. intramusculary two times a day.

Immediately after the end of the first course of phthorafurum the patient began taking gentian violet preparation, 3 mg per 50 kg of weight two times a day.

After taking taken the preparation for 3 days pain abated significantly, and then stopped completely. Hereinafter, pain has not returned.

### EXAMPLE 6

Female patient N., 64 years old. Diagnosis: compression body fracture L5. Pain syndrome. It is known from the anamnesis that after an accident the patient felt sharp burning pain in her lumbar, irradiating into her right leg. In CT, compression fracture of the fifth lumbar vertebra with wedge-shaped deformation was detected. Treatment and analgesic preparations were prescribed. Two months after the injury pain had abated, but for several years it has been resuming periodically with the same intensity. Analgesic preparations are ineffective. Crystal violet and malachite green oxalate were prescribed in a 1:1 ratio, with the dose of 4 mg per 70 kg of weight, two times a day. On the fifth day of taking the preparations the pain had abated, and after a week it disappeared completely. The preparations were taken for two more weeks. No resumption of pain after six months.

### EXAMPLE 7

Female patient T., 67 years old. Complaints of not being able to raise her right arm above the shoulder level. In CT, effects of osteoarthrosis with joint deformation were noticed. Taking analgesic preparations turned out to be ineffective. Pain became permanent. Gentian violet preparation was prescribed, 0.0004 mg/kg of weight once a day. After three days the pain had abated, and after a week it disappeared completely.

### EXAMPLE 8

Female patient O., 34 years old.

Complaints of periodic excruciating pain in the right temporal area. Diagnosis: migraine. Prescribed treatment: malachite green oxalate preparation, 2 g dissolved in 100 ml of water on an empty stomach two times a day. On the third day the pain abated and has not recurred.

### EXAMPLE 9

Male patient G., 43 years old. Diagnosis: complex regional pain syndrome (CRPS). Earlier diagnosis: reflex sympathetic dystrophy and upper right extremity causalgia. Pain had been bothering her for 6 months. In the beginning the pain was stopping after a sympathetic block. However, after two weeks sensations of burning, aching or rheumatic pain accompanied by sensory disorders appear. Sympathectomy had been suggested, but the patient categorically declined it. Treatment was prescribed with crystal violet and malachite green oxalate in the 1:1 ratio, 4 g/kg in the form of microclysters, two times a day. After two days pain disappeared and never recurred. The treatment continued for a week. Observation has demonstrated that the pain syndrome was arrested completely.

### EXAMPLE 10

A male patient with phantom pain after amputation of the right lower leg. Treatment using alcohol-novocain blocks was conducted repeatedly. However, despite the treatment the pain had become intolerable. Treatment was conducted with preparation rhodamine 6G, 0.000001 mg in saline (5 ml)—injection in the stump area. Injections were given two times a day. On the third day the pain abated, and after five days it disappeared. For the last 6 months pain has not recurred.

Thus, a medicinal agent has been found with pronounced analgesic effect in various chronic pain syndromes, including oncological diseases, which is also suitable as an analgesic in general anesthesia, and has no side effects.

Herein, one uses as analgesic agents widely available and inexpensive substances that are non-toxic, have no allergic effects and cause no addiction (dependence) when used in pharmaceutically acceptable doses; the treatment time is reduced, treatment efficiency is enhanced, and pain relief is provided while preserving patients' functional activity, using the minimum arsenal of analgesic agents with different effects, and completely eliminating the need for opiates.

1. (canceled)

2-9. (canceled)

10. The use of an organic dye from the group of crystal violet, gentian violet, malachite green oxalate and 6G as an analgesic agent in the form of a solution, with the agent dosage ranging from 0.000001 mg to 10 g per intake.

11. The use according to claim 1, wherein the agent is taken at least two times a day.

12. The use according to claim 2, wherein the agent is administered by mouth in the form of an aqueous solution.

13. The use according to claim 3, wherein the agent is taken before or after meals.

14. The use according to claim 2, wherein the agent is administered per rectum in the form of an aqueous solution.

15. The use according to claim 2, wherein a 1% solution is administered intravenously.

16. The use according to claim 2, wherein the agent is used in the form of a 1-5% ointment.

17. The use according to claim 2, wherein the agent is administered per rectum in the form of suppositories.

18. The use according to claim 2, wherein when arresting cancer pain the agent is injected into the tumor (under the tumor) or into a metastasis.

19. The use according to claim 10, wherein the agent is administered by mouth in the form of an aqueous solution.

20. The use according to claim 19, wherein the agent is taken before or after meals.

21. The use according to claim 10, wherein the agent is administered per rectum in the form of an aqueous solution.

22. The use according to claim 10, wherein a 1% solution is administered intravenously.

23. The use according to claim 10, wherein the agent is used in the form of a 1-5% ointment.

24. The use according to claim 10, wherein the agent is administered per rectum in the form of suppositories.

25. The use according to claim 10, wherein when arresting cancer pain the agent is injected into the tumor (under the tumor) or into a metastasis.
